(19) United States
(12) Patent Application Publication
(10) Pub. No.: US 2006/0084856 A1
(43) Pub. Date: Apr. 20, 2006

(54) Combination ophthalmic instrument

(76) Inventors: David Biggins, Colden, NY (US); Donald E. Miller, West Seneca, NY (US)

Correspondence Address: HODGSON RUSS LLP, ONE M & T PLAZA, SUITE 2000, BUFFALO, NY, 14203-2391, US

(21) Appl. No.: 10/969,658

(22) Filed: Oct. 20, 2004

(51) Int. Cl. A61B 3/16; A61B 5/103

(52) U.S. Cl. 600/399; 128/903; 600/587

(57) ABSTRACT

A combination ophthalmic instrument comprises a non-contact measurement system and a contact measurement system for measuring parameters of the eye. Measurement values from both systems are presented on a display of the instrument. A measurement value generated by one of the systems is automatically adjusted based on a measurement value generated by the other system in accordance with stored correction information.

### FIELD OF THE INVENTION

The invention relates to the field of ophthalmic instruments, and in particular to a combined ophthalmic instrument obtaining measurement signal information by both non-contact and contact measurement means.

### BACKGROUND OF THE INVENTION

Combined ophthalmic instruments capable of performing more than one type of ophthalmic measurement with respect to an eye of a patient are known. For example, U.S. Pat. No. 5,131,739 to Katsuragi discloses an ophthalmic instrument having non-contact tonometric measurement means for measuring intraocular pressure (IOP) using a fluid pulse, along with keratometer means for optically determining the corneal radius of curvature by projecting a predetermined target mark for reflection by the cornea. In another example, U.S. Pat. No. 6,193,371 teaches a combination ophthalmic instrument comprising two non-contact test means, namely an optical keratometer means combined with an optical pachymeter means.

It has been recognized for at least the past decade that tonometer measurements of IOP are influenced by corneal effects quantitatively represented by corneal thickness. See American Journal of Ophthalmology, May 1993, Volume 115, pages 592-596. Consequently, attempts have been made to provide a combined ophthalmic instrument capable of measuring both IOP and corneal thickness to allow for correction of the IOP measurement in view of the corneal thickness measurement. In particular, U.S. Pat. No. 5,474,066 to Grolman discloses a non-contact tonometer (NCT) having optical pachymeter means for measuring corneal thickness by slit illumination and image detection. While the NCT portion of the instrument was based on well-established technology, the incorporation of an optical system for measuring corneal thickness without contacting the eye was not accomplished in a commercially viable manner.

In another attempt described in U.S. Pat. No. 6,113,542 to Hyman et al., a contact applanation tonometer and a contact ophthalmic pachymeter having respective contact probes are connected to a shared microprocessor. To applicants' knowledge, this instrument has not found commercial acceptance, perhaps due in part to the burdens imposed on the patient and the operator in performing two contact measurements in succession.

As a result, there remains today a need for a commercially viable ophthalmic instrument capable of measuring both IOP and comeal thickness.

### SUMMARY OF THE INVENTION

The present invention meets the need set forth above by combining non-contact and contact measurement means in one instrument.

An embodiment of a combination ophthalmic instrument formed in accordance with the present invention generally comprises non-contact measurement means for generating first measurement signal information without contacting the eye, contact measurement means for generating second measurement signal information by contacting the eye, signal processing means for evaluating the first signal information to provide a first measurement value and for evaluating the second signal information to provide a second measurement value, and a display connected to the signal processing means for displaying the first and second measurement values. More particularly, a described embodiment comprises a non-contact tonometer having a tonometer control microprocessor, a contact pachymeter having a pachymeter control microprocessor, a main control microprocessor connected to the tonometer control microprocessor and to the pachymeter control microprocessor, a memory device connected to the main control microprocessor, and a display connected to the main control microprocessor, whereby measurement values obtained by the non-contact tonometer and by the contact pachymeter are presented on the display. Preferably, an adjusted IOP value is computed based on a raw IOP measurement value obtained by the non-contact tonometer and a corneal thickness measurement value obtained by the contact pachymeter using stored correction information.

The non-contact tonometer and display are housed by a main housing of the instrument. The contact pachymeter preferably includes a hand-held probe movable separately from the main housing and carrying an ultrasonic transducer. Electronics of the instrument, including the mentioned microprocessors, are housed within the main housing. A graphic user interface presented on the display includes icons corresponding to command buttons on the main housing for menu-driven user operation.

### BRIEF DESCRIPTION OF THE DRAWINGS

The nature and mode of operation of the present invention will now be more fully described in the following detailed description taken with the accompanying drawing figures, in which:

FIG. 1 is a perspective view of a combination ophthalmic instrument formed in accordance with an embodiment of the present invention.

FIG. 2 is another perspective view of the combination ophthalmic instrument shown in FIG. 1, partially sectioned to show the arrangement of circuit boards within a main housing of the instrument;

FIG. 3 is a schematic block diagram showing electronic circuitry of the combination ophthalmic instrument shown in FIGS. 1 and 2;

FIG. 4 is a schematic diagram showing electronic circuitry associated with a contact measurement means of the combination ophthalmic instrument;

FIG. 5 shows a measure screen of a graphic user interface of the present invention prior to right eye measurement;

FIG. 6 shows the measure screen of the graphic user interface after a first IOP measurement has been taken on the right eye;

FIG. 7 shows the measure screen of the graphic user interface after three IOP measurements have been taken on the right eye;

FIG. 8 shows the measure screen of the graphic user interface after three IOP measurements have also been taken on the left eye;

FIG. 9 shows a pachymeter screen of the graphic user interface prior to measurement of corneal thickness;

FIG. 10 shows the pachymeter screen of the graphic user interface after the right eye has been selected by a user for measurement of comeal thickness;

FIG. 11 shows the pachymeter screen of the graphic user interface after the corneal thickness of the right eye has been measured; and

FIG. 12 shows the pachymeter screen of the graphic user interface after the corneal thickness of the left eye has also been measured.

### DETAILED DESCRIPTION OF THE INVENTION

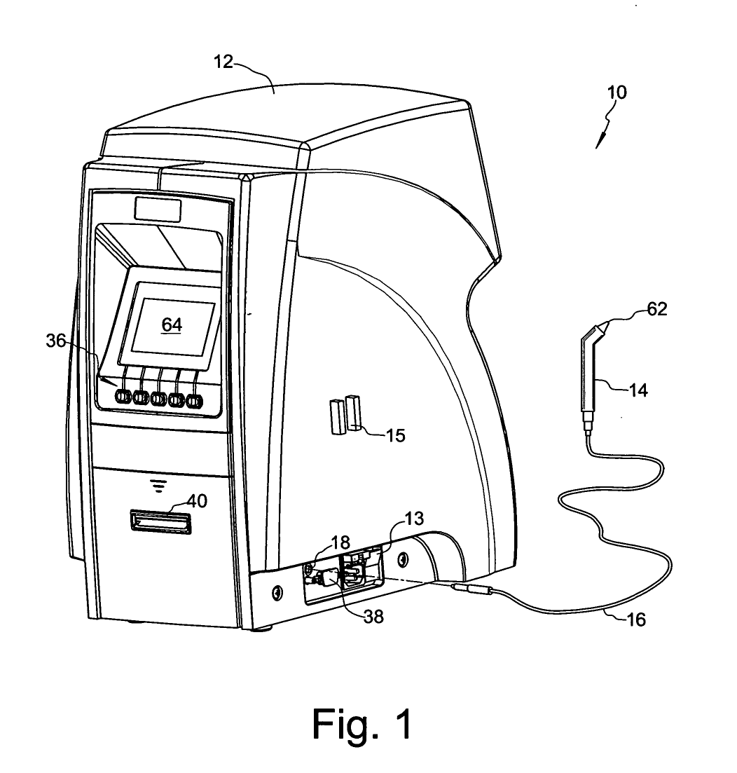

FIGS. 1-3 illustrate a combination ophthalmic instrument 10 formed in accordance with a preferred embodiment of the present invention. Ophthalmic instrument 10 generally comprises a main housing 12 and a hand-held probe 14 connected to the main housing by a flexible conductive cable 16 plugged into a connection port 18 in main housing 12. A mounting means 15 is provided on main housing 12 for removeably attaching the probe 14 to the main housing. Mounting means 15 is shown to be a pair of opposed, elastically deformable tabs on a sidewall of main housing 12 for gripping probe 14 by friction, however many other forms are possible, including without limitation hooks, male-female plug arrangements, and hook-and-loop fabric. A power switch: 13 is provided on main housing 12. Also visible in FIG. 1 is a set of control buttons 36, a serial communications port 38, a printer 40, and a color display 64 each carried by main housing 12.

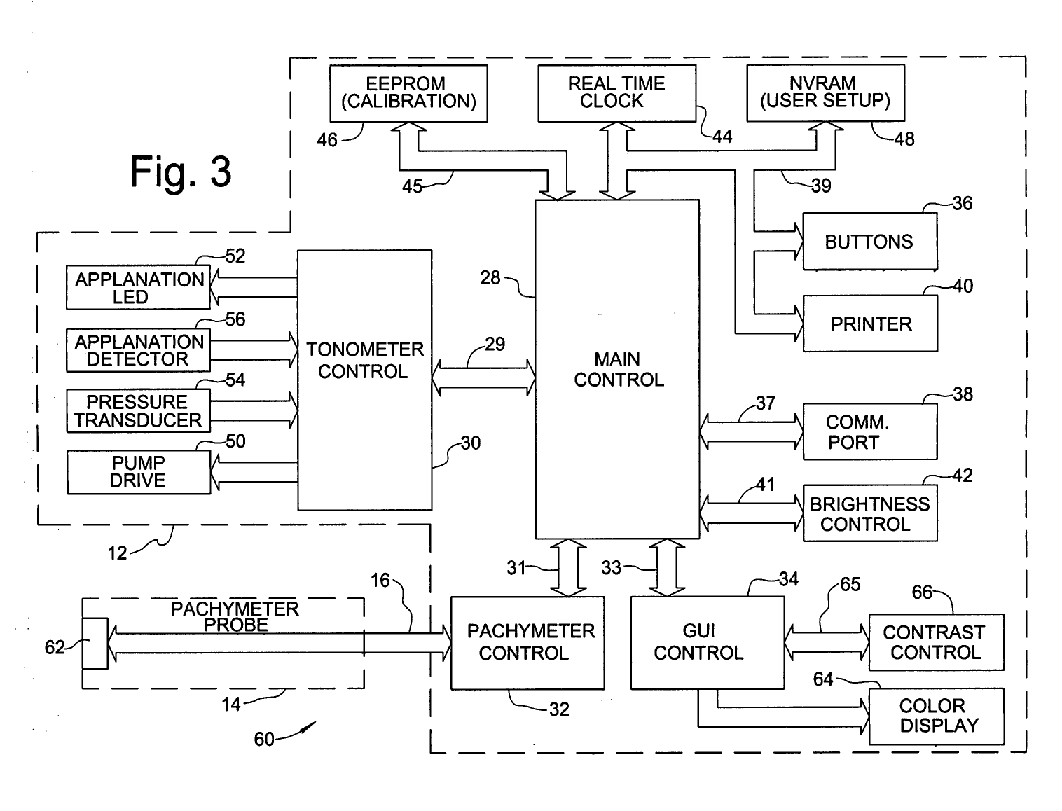

Main housing 12 houses a non-contact measurement means 20 for generating ophthalmic measurement signal information without contacting the eye. In the embodiment now being described, non-contact measurement means 20 comprises a non-contact tonometer for measuring intraocular pressure of a patient's eye by directing a fluid pulse at the eye to transfigure the cornea, as is well known in the art of ophthalmic instruments. Accordingly, non-contact measurement means 20 includes an electromechanical pump energized by a pump drive 50 for generating the fluid pulse, a pressure transducer 54 associated with a plenum chamber of the pump for sensing fluid pressure within the plenum chamber, an applanation LED 52 for emitting illumination directed at the cornea, and an electro-optical applanation detector 56 arranged to receive corneally reflected light to provide a signal indicating applanation status of the comea. A tonometer control microprocessor 30 communicates with the pump drive 50, applanation LED 52, pressure transducer 54, and applanation detector 56 as shown in FIG. 3 to provide control commands and receive pressure signal information from pressure transducer 54 and applanation signal information from applanation detector 56.

The signal information from pressure transducer 54 and applanation detector 56 is evaluated by tonometer control microprocessor 30 to provide a first measurement value indicative of intraocular pressure. The first measurement value is stored in internal memory of tonometer control microprocessor 30 and communicated in digital signal form to a main control microprocessor 28 of instrument 10.

A suitable main housing 12, non-contact measurement means 20, tonometer control microprocessor 30, and main control microprocessor 28 are found in the model AT-555 Non-Contact Tonometer and the ORA™ Ocular Response Analyzer, both of which are available from Reichert, Inc. of Depew, N.Y., assignee of the present application and invention. The specific form of the non-contact measurement means 20 is open to wide variation, and may include a non-contact tonometer measurement system different from that found in the AT-555 Non-Contact Tonometer and ORA™ Occular Response Analyzer. In the context of the present invention, all other commercially available non-contact tonometers—past, present, and future—are deemed to provide non-contact measurement means equivalent to the means expressly disclosed in this specification, and may be used as a foundation for practicing the present invention.

A contact measurement means 60 for generating ophthalmic measurement signal information by contacting the eye is carried in part by hand-held probe 14. In the present embodiment, contact measurement means 60 comprises an ultrasonic pachymeter for measuring corneal thickness of the eye, such means already being known in the field of ophthalmology. Contact measurement means 60 is shown as including an ultrasonic transducer 62 carried by probe 14 and operable to provide signal information when the transducer is placed in contact with the cornea. A pachymeter control microprocessor 32 within main housing 12 communicates with transducer 62 over cable 16 to provide control commands and receive signal information from the transducer.

The signal information provided by transducer 62 is received and evaluated by pachymeter control microprocessor 32 to yield a second measurement value indicative of corneal thickness that is stored in internal memory of pachymeter control microprocessor 32. When called for, the second measurement value is communicated in digital signal form to main control microprocessor 28. Pachymeter probes suitable for practicing the present invention are currently sold by Blatek, Inc. of State College, Pennsylvania under model numbers AT15387 and AT15399.

It is emphasized that the present invention can be practiced using other commercially available pachyrneter probes, or a pachymeter probe designed in the future. For example, DGH Technology, Inc., Haag-Streit AG, and Portable Ophthalmic Devices, Inc. currently offer pachymeter probes capable of being used in practicing the present invention. In the context of the present invention, all other commercially available pachymetric contact probes—past, present, and future—are deemed useful in providing contact measurement means equivalent to the means expressly disclosed in this specification, and may be used in practicing the present invention.

Those skilled in the art will recognize that control signals to, and measurement signal information from, transducer 62 can be transmitted to pachymeter control microprocessor 32 in main housing 12 by way of wireless communication protocols, assuming that suitable transceiver hardware and software is provided.

The schematic block diagram of FIG. 3 generally illustrates the arrangement and interconnection of electronic components of ophthalmic instrument 10. The main housing 12 and probe 14 are represented in dashed line. Main housing 12 houses main control microprocessor 28, tonometer control microprocessor 30, pachymeter control microprocessor 32, and a graphic user interface (GUI) control microprocessor 34. A commercially available microprocessor suitable for use as main control microprocessor 28 is the MC68306 integrated processor from Motorola, Inc. Tonometer control microprocessor 30 is preferably a Hitachi H8 microcontroller connected to main control microprocessor 28 by an I2C bus 29. Both the Motorola MC68306 and the Hitachi H8 are currently used in the aforementioned AT-555 Non-Contact Tonometer from Reichert, Inc. In the present embodiment, pachymeter control microprocessor 32 is preferably an MC9328MX1 (Dragonball™ MX1) system processor from Motorola, Inc. that communicates with main control microprocessor 28 over an I2C bus 31. GUI control microprocessor 34 is preferably a Dragonball™ MX1 processor as well, and is connected to main control microprocessor 28 by serial communications bus 33. While suitable microprocessors are specifically identified above, other microprocessors may be used in practicing the invention.

The main housing 12 of ophthalmic instrument 10 further houses control buttons 36, serial communications port 38, and printer 40. Control buttons 36 are connected to main control microprocessor 28 by an address/data bus 39 and are positioned directly below display 64 to correspond with display icons appearing in menu screens of the GUI as described in greater detail below. Serial communications port 38 is connected to main control microprocessor 28 by a serial communications bus 37 and enables connection of an external device such as a personal computer. Printer 40 is connected to main control microprocessor 28 by address/data bus 39, and may be conveniently embodied as a thermal printer internally mounted in housing 12. A brightness control 42 for adjusting brightness of display 64 is connected to main control microprocessor 28 by an I2C bus 41. Display 64 is preferably a color liquid crystal display, however the term “display” is intended to mean any electronic display device.

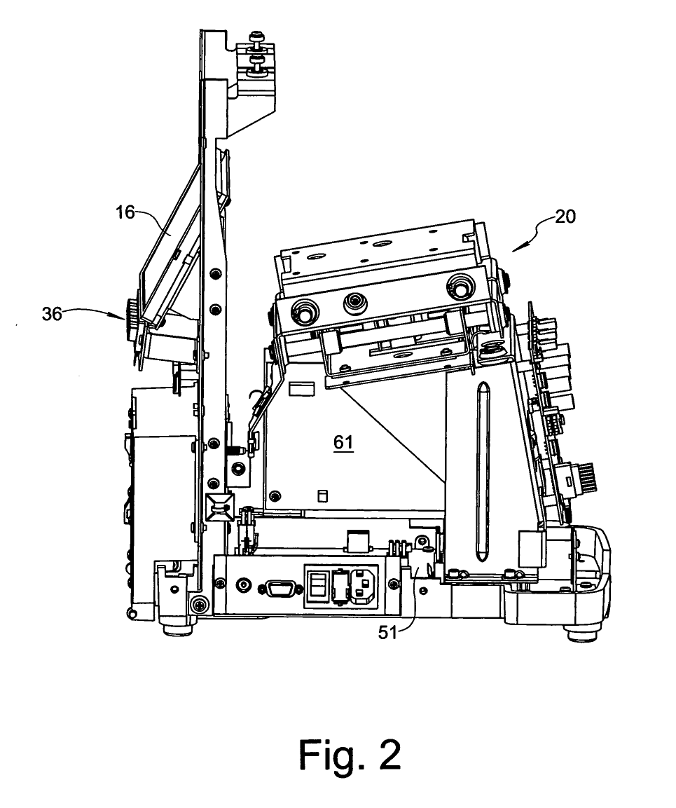

Additional electronic modules connected to main control microprocessor 28 and residing within housing 12 include a real time clock 44, non-volatile RAM 48 for storage of user setup data and possibly measurement data, and an EEPROM 46 for storage of calibration data. Clock 44 and NVRAM 48 communicate with main control microprocessor 28 over address/data bus 39, while EEPROM 46 communicates with main control microprocessor 28 over an I2C bus 45. As can be seen in FIG. 2, a first printed circuit board 51 is mounted near the base of main housing 12 and includes main control microprocessor 28, GUI control microprocessor 34, and memory and circuitry not specifically associated with contact measurement means 60.

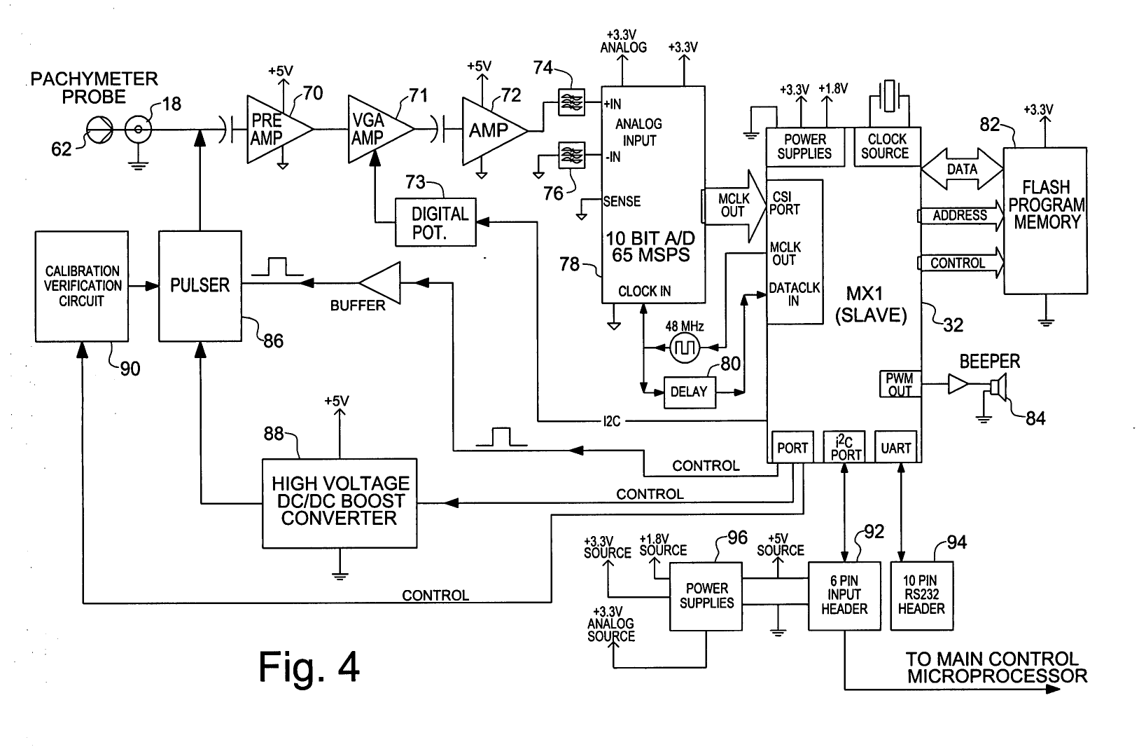

Electronics associated with contact measurement means 60 are provided on a second printed circuit board 61 (FIG. 2) in main housing 12 and are illustrated in FIG. 4. The analog signal from ultrasonic transducer 62 is amplified by a preamplifier 70, adjustable gain amplifier 71, and differential amplifier 72. A digital potentiometer 73 connected to an I2C port of main control microprocessor 28 and to adjustable gain amplifier 71 facilitates replacement of the ultrasonic transducer 62 in the field, in the event replacement becomes necessary. The amplified analog signal is processed by frequency filters 74 and 76 to provide a well-defined analog input signal to an analog-to-digital converter 78. Digital data are output from A/D converter 78 to a channel state information (CSI) port of pachymeter control microprocessor 32. In a preferred implementation, A/D converter 78 is a ten-bit converter, and only eight bits are used (the lowest two bits are discarded as noise). Data sampling from A/D converter 78 is driven by a 48 MHz clock pulse subject to a delay gate 80. A flash programmable memory device 82 is connected to pachymeter control microprocessor 32 for storing pachymeter control software. A beeper 84 connected to a pulse-width modulation module of pachymeter control microprocessor 32 provides an audible signal when measurement of an eye is completed.

Transducer 62 is excited by narrow square-wave pulses generated by pulser 86, which receives control signals from pachymeter control microprocessor 32. A high voltage DC/DC boost converter 88 is connected to provide voltage potential across a piezoelectric element of ultrasonic transducer 62, whereby the excitation pulses from pulser 86 trigger acoustic output by transducer 62. A calibration verification circuit 90 is provided between pachymeter control microprocessor 32 and pulser 86, whereby pulses of known frequency can be introduced for calibration purposes.

Also shown in FIG. 4 is a six-pin input header 92 connected to an 12C port of pachymeter control microprocessor 32. Input header 92 is used for connecting pachymeter control microprocessor 32 to main control microprocessor 28. A ten-pin RS232 header 94 is also provided for temporarily connecting an external computer to upload and download programming code. Power supply circuits are represented at block 96.

The combination ophthalmic instrument 10 of the present invention allows measurement values taken with one type of measurement means to be adjusted or corrected based on measurement values taken with another type of measurement means. In the embodiment now described, measurement values taken using non-contact measurement means 20 can be adjusted or corrected based on measurement values taken using contact measurement means 60. Specifically, correction information stored by internal memory of main control microprocessor 28 enables main control microprocessor 28 to calculate a corrected IOP value from the originally measured IOP value based on the measured corneal thickness. The correction information can be in the form of a correction data table or a correction function. For example, the measured IOP value can be adjusted according to data published by Ehlers et al. (1975) as modified by Stodmeister (1998), assuming a mean corneal thickness in healthy subjects of 545 μm as in accordance with Doughty and Zaman (2000). This correction data table is reproduced below:

| | CORRECTION VALUE ADDED TO |
|---|---|
| CORNEAL THICKNESS (μm) | MEASURED IOP (mmHg) |
| 445 | +7 |
| 455 | +6 |
| 465 | +6 |
| 475 | +5 |
| 485 | +4 |
| 495 | +4 |
| 505 | +3 |
| 515 | +2 |
| 525 | +1 |
| 535 | +1 |
| 545 | 0 |
| 555 | −1 |
| 565 | −1 |
| 575 | −2 |
| 585 | −3 |
| 595 | −4 |
| 605 | −4 |
| 615 | −5 |
| 625 | −6 |
| 635 | −6 |
| 645 | −7 |

The correction stored in memory may be fixed, and need not be stored in the internal memory of main control microprocessor 28. For example, the correction information could instead be stored by internal memory on pachymeter control microprocessor 32. It is also contemplated to program the GUI control microprocessor 34 to enable a user to customize or change the stored correction data table and/or correction formula as new studies are published. For this purpose, it is advantageous to store the correction information in NVRAM 48 rather than in the internal memory of main control microprocessor 28 or pachymeter control microprocessor 32.

The combination ophthalmic instrument of the present invention allows a user to conveniently operate both non-contact and contact measurement means using the same control buttons 36 and display 64 associated with main housing 12. Also, the invention allows the user to view and print measurement values using the same display 64 and printer 40. With respect to adjustment or correction of measured IOP, there is no need to manually enter a comeal thickness value to use as a basis for correction, as this value is automatically stored and used by instrument 10.

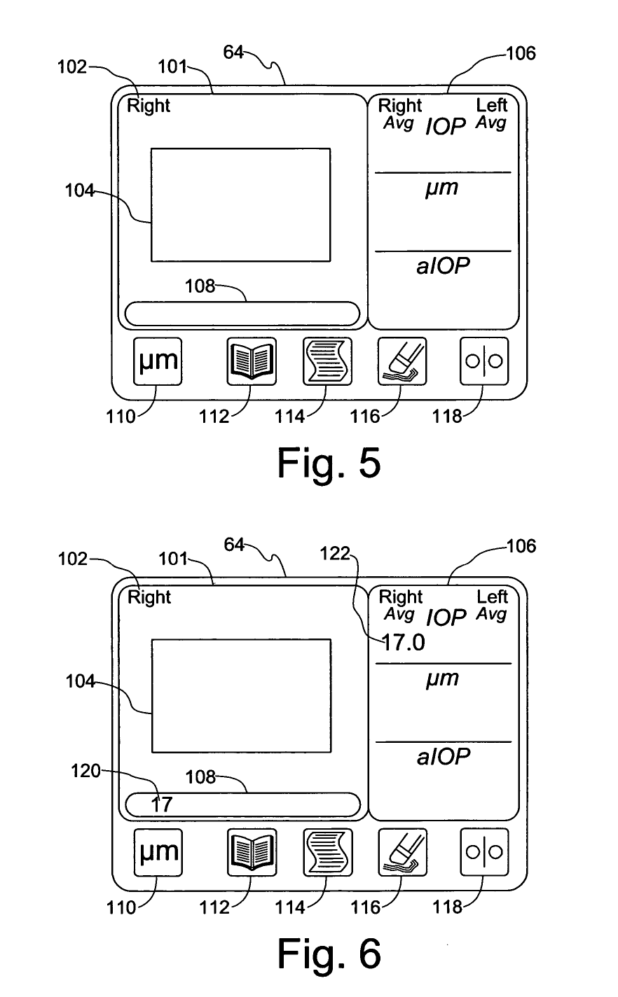

The manner of using the invention will now be described in connection with an embodiment based on the aforementioned AT-555 Non-Contact Tonometer by Reichert, Inc., wherein an updated GUI is provided to accommodate pachymetric measurements in addition to tonometric measurements. In this regard, reference is now made to FIGS. 5-12, which show various display screens of the GUI. It will be understood that the display icons appearing in the display screens are selectable by a user by pressing one of the command buttons 36 located directly beneath the corresponding display icon.

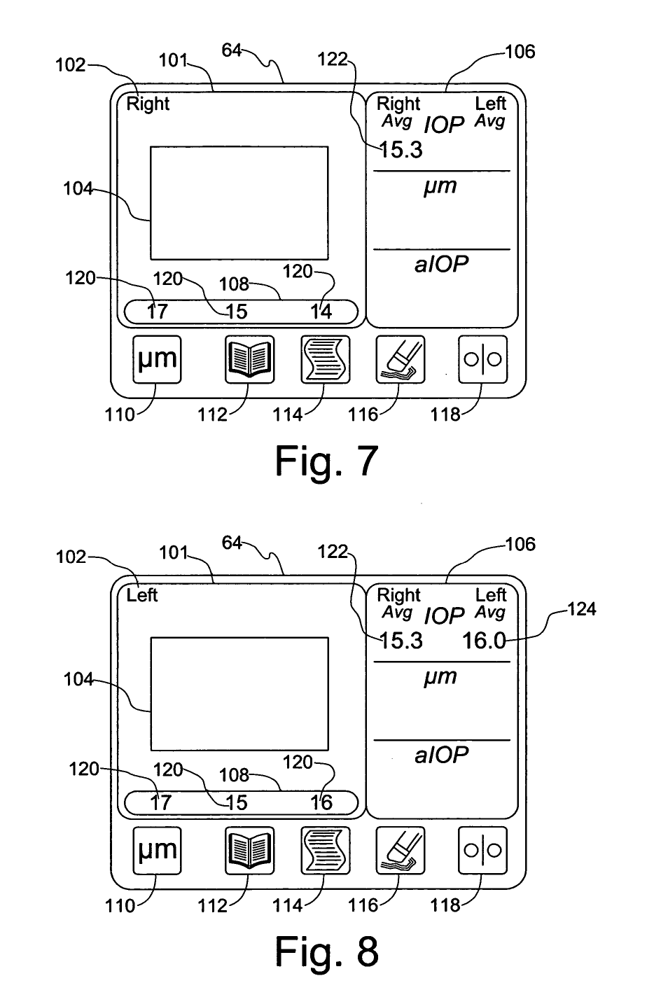

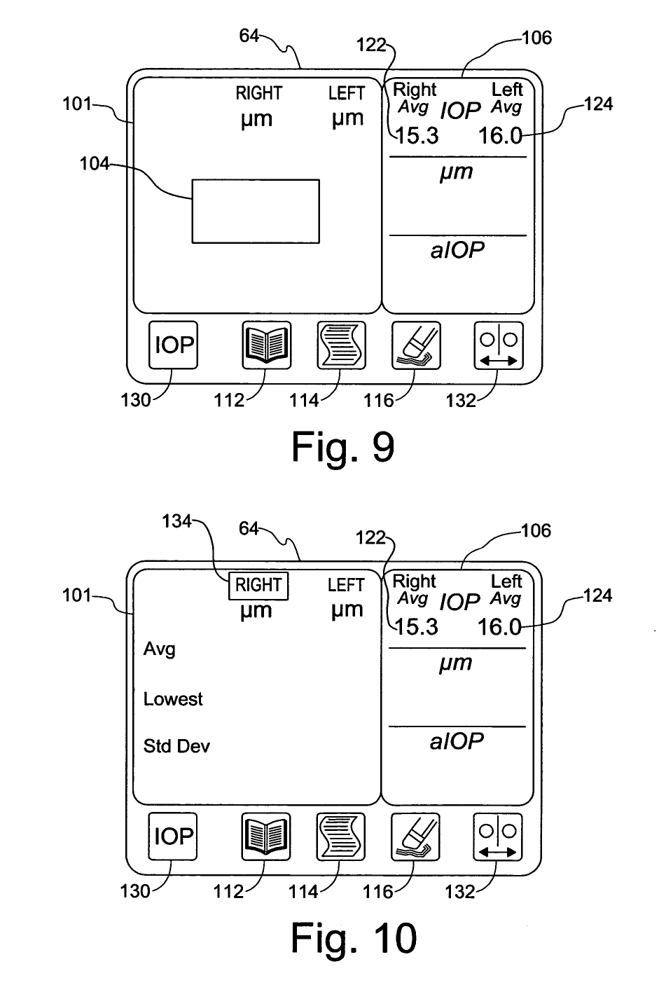

FIG. 5 shows a measure screen of the GUI as it appears on display 64. The measure screen preferably includes a main panel 101, a left/right indicator 102 at the upper left corner of the main panel which indicates whether a patient forehead rest (not shown) of the instrument is positioned for left eye or right eye measurement, a pop-up message box 104 in the main panel for displaying text messages to the user, a results panel 106 for displaying various measurement results, and a report bar 108 in the main panel for displaying individual IOP measurement values as they are taken. Measure screen further includes five display icons respectively corresponding to command buttons 36, namely a pachymeter icon 110 selectable to display a pachymeter screen (see FIGS. 9-12), a review icon 112 selectable to display a review screen, a print icon 114 selectable to print measurement results on printer 40, an erase icon 116 selectable to clear all measurement results from memory, and a measure icon 118 selectable to initiate automatic alignment of non-contact measurement means 20 with the eye followed by IOP measurement (in this case the right eye is measured first). After the command button 36 corresponding to measure icon 118 has been pressed, 5 automatic alignment is carried out, a tonometer measurement is taken, and an IOP measurement value 120 is displayed in report bar 108 as shown in FIG. 6. In addition, an average IOP value 122 for the right eye is calculated and displayed in results panel 106. As further IOP measurements are taken in this way, the IOP measurement values 120 appear in report bar 108 and the average IOP value 122 is recalculated and displayed in results panel 106, as can be understood from FIG. 7. To measure IOP of the other eye, the forehead rest is shifted laterally such that left/right indicator 102 changes, in this case from “Right” to “Left” as shown in FIG. 8, and the command button 36 corresponding to measure icon 118 is pressed in the manner described above. The IOP measurement values 120 are displayed in report bar 108 and the average IOP valued 124 for the left eye is displayed in results panel 106.

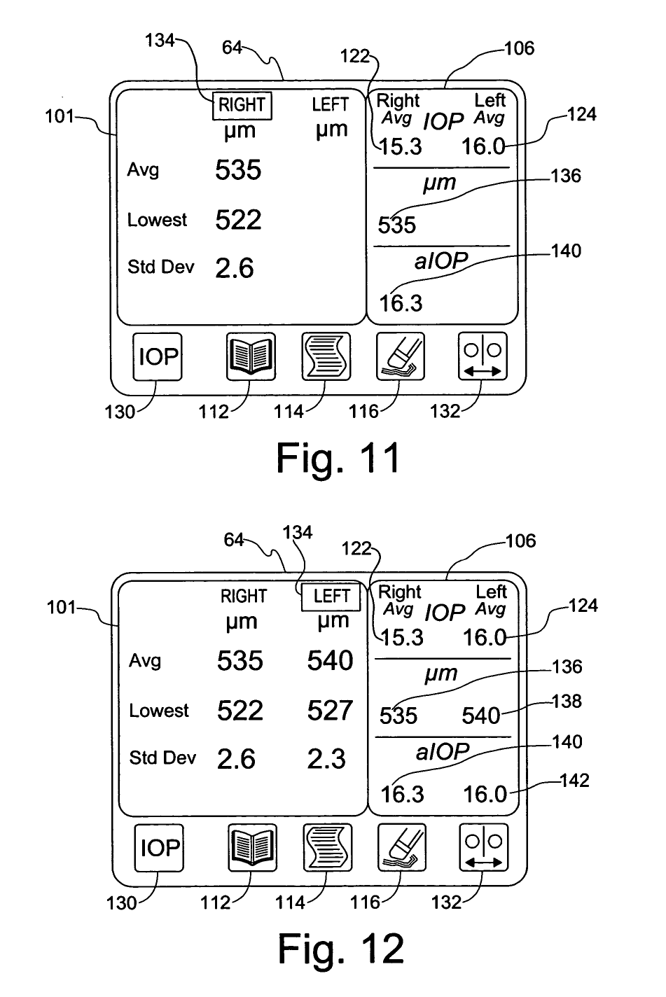

The user may switch over from the measure screen to a pachymeter screen of the GUI by pressing the command button 36 corresponding to pachymeter icon 110. The pachymeter screen, shown in FIG. 9, is generally similar to the measure screen but includes a tonometer icon 130 replacing the pachymeter icon 110 and an eye select icon 132 replacing measure icon 118. Also, the report bar 108 is removed and current measurement values are displayed in main panel 101. To select an eye for measurement by contact measurement means 60, the user presses the command button 36 corresponding to eye select icon 132, and a highlight bar 134, shown in FIG. 10, highlights either “RIGHT” or “LEFT” in main panel 101 in the manner of a toggle selector. The user then manually moves probe 14 to place ultrasonic transducer 62 into contact with the cornea of the selected eye, and measures comeal thickness in a known manner depending upon the specific type of probe being used. The signal information from transducer 62 is communicated to pachymeter control microprocessor 32, and ultimately a pachymetric measurement value is calculated and displayed. Actually, a large number of comeal thickness readings are taken in rapid succession, and the average of the readings is displayed as a comeal thickness value 136 in results panel 106 and in main panel 101. The lowest reading and standard deviation of the readings are also displayed in main panel 101. As will be understood by reference to FIG. 11, if an IOP value has previously been obtained for the eye, then an adjusted IOP or “aIOP” value 140 is calculated using the stored correction information and displayed in results panel 106. The user may then toggle over to the other eye by pressing the command button corresponding to eye select icon 132, causing highlight bar 134 to move accordingly, and repeat the pachymetric measurement process on the other eye as indicated by FIG. 12. The user returns to the measure screen from the pachymeter screen by pressing the command button 36 corresponding to tonometer icon 130.

It is noted that the order of measurement as between the non-contact and contact measurement means is not critical, and can be reversed from the order described above. It is also noted that a direct touch display screen may be used to allow a user to directly interact with the screen icons, rather than using command buttons 36.

While preferred embodiments of the present invention have been disclosed, it will be appreciated that the present invention can be otherwise embodied within the scope of the following claims.

What is claimed is:

1. An ophthalmic instrument for testing an eye, the instrument comprising:
non-contact measurement means for generating first measurement signal information without contacting the eye;
contact measurement means for generating second measurement signal information by contacting the eye;
signal processing means for evaluating the first signal information to provide a first measurement value and for evaluating the second signal information to provide a second measurement value; and
a display connected to the signal processing means for displaying the first and second measurement values.

2. The ophthalmic instrument according to claim 1, further comprising:
a hand-held probe carrying a portion of the contact measurement means; and
a main housing carrying the non-contact measurement means, the signal processing means, and the display;
wherein the probe is manually movable relative to the main housing.

3. The ophthalmic instrument according to claim 2, wherein a flexible cable connects the probe to the main housing and transmits the second measurement signal information.

4. The ophthalmic instrument according to claim 2, wherein the second measurement signal information is transmitted from the probe to the main housing by wireless communication.

5. The ophthalmic instrument according to claim 2, wherein the main housing includes mounting means for removeably attaching the probe thereto.

6. The ophthalmic instrument according to claim 1, wherein the first measurement value is indicative of intraocular pressure.

7. The ophthalmic instrument according to claim 1, wherein the second measurement value is indicative of corneal thickness.

8. An ophthalmic instrument for testing an eye, the instrument comprising:
non-contact measurement means for generating first measurement signal information without contacting the eye;
contact measurement means for generating second measurement signal information by contacting the eye;
a memory means for storing correction information; and
signal processing means for evaluating the first signal information to provide a first measurement value, and for evaluating the second signal information to provide a second measurement value;
wherein the signal processing means is connected to the memory means and is programmed to correct the first measurement value using the second measurement value and the stored correction information.

9. The ophthalmic instrument according to claim 8, wherein the correction information includes a correction data table.

10. The ophthalmic instrument according to claim 8, wherein the correction information includes a correction function.

11. The ophthalmic instrument according to claim 8, wherein the memory means is programmable to permit the correction information to be changed.

12. An ophthalmic instrument comprising:
a non-contact tonometer;
a contact pachymeter
a main control microprocessor connected to the non-contact tonometer and to the pachymeter;
a memory device connected to the main control microprocessor; and
a display connected to the main control microprocessor;
whereby measurement values obtained by the non-contact tonometer and by the contact pachymeter are presented on the display.
